(19) United States
(12) Patent Application Publication
(10) Pub. No.: US 2023/0040943 A1
(43) Pub. Date: Feb. 9, 2023

(54) MEDICAMENT COMPRISING A COMBINATION OF AMNIOTIC MEMBRANE AND UMBILICAL CORD BLOOD SERUM FOR WOUND HEALING

(71) Applicant: PLACELTA (PTY) LTD, Johannesburg (ZA)

(72) Inventors: Carl MONTAGUE, Kloof (ZA); Yvonne HOLT, Hyde Park (ZA)

(73) Assignee: PLACELTA (PTY) LTD, Johannesburg (ZA)

(21) Appl. No.: 17/759,302

(22) PCT Filed: Jan. 21, 2021
(86) PCT No.: PCT/IB2021/050440

§ 371 Date: Jul. 22, 2022

(30) Foreign Application Priority Data
Jan. 24, 2020 (NL) 2024751

(51) Int. Cl. A61K 35/50; A61K 9/70; A61K 35/51; A61K 38/18; A61K 38/20; A61K 38/19; A61K 38/21; A61K 38/17; A61P 17/02

(52) U.S. Cl. CPC A61K 35/50; A61K 9/7007; A61K 35/51; A61K 38/18; A61K 38/20; A61K 38/191; A61K 38/193; A61K 38/202; A61K 38/208; A61K 38/212; A61K 38/1793; A61K 38/1808; A61K 38/1833; A61K 38/2006; A61K 38/2086; A61P 17/02

(57) ABSTRACT

The invention is directed at a medicament comprising a combination of dehydrated or cryopreserved amniotic membrane isolated from human or other mammalian placenta, wherein the amniotic membrane is in a form of a sheet or a sheet composite comprising multiple overlain sheets of amniotic membrane; and umbilical cord blood serum, isolated from umbilical cord blood of a human or other mammal, separately of each other, for combined use in a method of treating a tissue wound in a human or other mammalian subject by applying the amniotic membrane to injured tissue of the wound to contact injured tissue of the wound and contacting the amniotic membrane with the umbilical cord blood serum.

### FIELD OF THE INVENTION

THIS INVENTION relates to the treatment of tissue wounds in mammals. The invention provides for the use of amniotic membrane, obtained from human or other mammalian placenta, and umbilical cord blood serum, for the treatment of such wounds.

### BACKGROUND TO THE INVENTION

Wound healing is characterized as a biologic process involving a series of steps including haemostasis, inflammation, cellular proliferation, and tissue remodelling. Often an injury will result in haemostasis when the blood clots in an attempt to stem blood loss. Following this, the innate immune response leads to the recruitment of macrophages and neutrophils to the site of the wound and the stimulation of an inflammatory response. The proliferative phase leads to the development of new tissue and extracellular matrix (ECM) which, during the maturation phase, is remodelled and rearranged.

Chronic or non-healing wounds develop when there is dysregulation of the wound healing processes. This can be due to systemic disorders such as inflammation, diabetes, malnutrition, metabolic diseases, and immunosuppression. Aging is also associated with delayed wound healing. Often chronic wounds will not heal properly, and therapeutic intervention is required to close the wound and/or promote healing. Chronic wounds are a significant health care problem and may have serious implications for quality of life when they do not heal and require therapeutic intervention.

Many scientific studies have investigated the growth factors involved in the regulation of wound healing during the stages of haemostasis, inflammation, proliferation and remodelling. Some of the most important growth factors with regards to wound healing are epidermal growth factor (EGF), transforming growth factors (TGF), hepatocyte growth factor (HGF), vascular endothelial growth factor (VEGF), platelet derived growth factor (PDGF), fibroblast growth factors (FGF), and keratinocyte growth factor (KGF).

Amniotic membrane allografts have been used as a biologic therapy to promote soft tissue healing in ophthalmic and other peripheral wounds. Amniotic membrane consists of a combination of tissue and cells which, when used as biological dressing, promote re-epithelialisation and wound healing by acting as a foundation for the re-growth of soft tissue. A key property of amniotic membrane that contributes to its ability to promote wound healing is the provision of a scaffold of pre-formed extracellular matrix composed of natural collagen and other proteins, including fibronectin, laminins, proteoglycans and glycosaminoglycans.

The therapeutic benefits of amniotic membrane include: provision of a natural biological barrier to external insults; anti-microbial properties; anti-inflammatory properties; anti-scarring properties; prevents overgrowth of blood vessels; analgesic properties to reduce pain at site of injury; non-immunogenic and low antigenicity; protects against loss of fluids and proteins; and promotes epithelial cell growth. Amniotic membrane includes varying levels of biologically active cells in the epithelial and stromal layers that deliver growth factors and cytokines with anti-inflammatory, anti-bacterial, anti-immunogenic and anti-fibrotic properties, however the processing and storage of the amniotic membrane prior to grafting does significantly reduce these biologically active components. The invention addresses how to increase the levels of growth factors and cytokines by combining the administration of prepared amniotic membrane with umbilical cord blood serum in order to enhance the wound healing activity.

Blood-derived products have been used for the treatment of ocular surface diseases for many years as blood serum contains comparable levels and types of growth factors, cytokines and vitamins to natural tears. These blood-derived products include eye drops prepared either from the patient’s own blood serum (autologous serum) or from donors, such as allogeneic umbilical cord blood serum and allogeneic peripheral blood serum.

In the eye, persistent corneal epithelial defects can be the result of tear film abnormalities, intrinsic epithelial and basement membrane disorders, lid abnormalities, metabolic disturbances, iatrogenic disorders, trauma, chemical burns, immunological disorders, neurogenic disorders, nutritional disorders, infectious disorders and inflammation. Umbilical cord serum eye drops have been shown to be effective in the treatment of ocular surface disorders such as neurotrophic keratitis, severe dry eye, persistent epithelial defects, and recurrent corneal erosions. Umbilical cord blood serum eye drops are more effective than autologous serum eye drops in healing epithelial defects. Its efficacy has been demonstrated in various conditions, ranging from severe dry eye with or without primary Sjogren syndrome to ocular graft-versus-host-disease persistent epithelial defects, recurrent corneal erosions, chemical burns, and neurotrophic keratitis.

Umbilical cord blood serum contains growth factors that are beneficial to the healing of persistent corneal epithelial defects. These factors include EGF, FGF, PDGF, HGF, vitamin A, TGF-β, and nerve growth factor (NGF). The concentrations of EGF, TGF-β and NGF are much higher in umbilical cord serum than in peripheral blood serum.

### DISCLOSURE OF THE INVENTION

BROADLY, THE PRESENT INVENTION PROVIDES a combination therapy, and associated medicaments and the production thereof, comprising amniotic membrane, which provides an extracellular matrix scaffold to promote tissue wound healing, and umbilical cord blood serum, which provides a high concentration of growth factors and cytokines to promote wound healing.

The meaning of the term “medicament” as used herein includes a substance or composition, or a combination of two or more substances and/or compositions, useful for medical treatment or therapy. Within this meaning is included amniotic membrane in combination with umbilical cord blood serum, separately of or in contact with each other, as characterised herein.

Furthermore, the meaning of the term “treatment” as used herein includes therapeutic treatment, to promote wound healing in a subject.

Broadly, the combination therapy of the invention may include, in use, applying amniotic membrane, as characterised herein, to a wound, i.e. locating it in contact with a wound, specifically injured tissue of the wound, e.g. such that it covers the wound, in conjunction with applying, simultaneously or separately, e.g. sequentially, umbilical cord blood serum, as characterised herein, to the wound, and more specifically to the amniotic membrane contacting the wound.

The invention, therefore, in one form, provides a medicament for use in therapy, wherein the medicament comprises a combination of amniotic membrane and umbilical cord blood serum for treating tissue wounds, e.g. chronic or non-healing wounds. This combination has surprisingly been found to promote correcting or improvement of dysfunctions in wound healing processes, to stimulate healing of wounds, and decrease scarring.

ACCORDING TO A FIRST ASPECT OF THE INVENTION THERE IS PROVIDED a medicament comprising amniotic membrane and umbilical cord blood serum.

The medicament may therefore comprise a combination of amniotic membrane and umbilical cord blood serum. Typically, in the medicament, the amniotic membrane and umbilical cord blood serum would be provided separately of each other.

The amniotic membrane may be amniotic membrane isolated from human or other mammalian placenta.

The amniotic membrane may comprise isolated amniotic membrane in dehydrated form or in cryopreserved form. In other words, the amniotic membrane may comprise isolated amniotic membrane, isolated from human or other mammalian placenta, that has been dehydrated or cryopreserved.

In this regard, in one embodiment of the invention, no further processing steps, except perhaps sterilisation and/or removal of the epithelial layer in the case of dehydration, would typically have been undertaken on the membrane, except optionally to form it into a desired configuration, as hereinafter described.

Thus, in such an embodiment of the invention, the amniotic membrane may simply comprise dehydrated amniotic membrane as a sheet of amniotic membrane, as isolated from human or other mammalian placenta, that has been subjected to dehydration or cryopreservation.

The amniotic membrane may have its extracellular matrix intact.

As alluded to above, the epithelial cell layer of the amniotic membrane may have been removed in the case of a dehydrated amniotic membrane. This would not necessarily be the case of a cryopreserved amniotic membrane, in which form the epithelial cell layer may have been retained.

The amniotic membrane may be in the form of a sheet of amniotic membrane.

The amniotic membrane sheet may be provided, optionally prepared, as a single sheet of amniotic membrane, or multiple overlain sheets of amniotic membrane may be provided as an amniotic membrane sheet composite. In the latter respect, the sheets of such a composite may, for example, be laminated together, e.g. by means of pressing. Typically, such a composite may comprise up to five sheets of amniotic membrane.

Hereinafter, reference to “sheet” includes both a single amniotic membrane sheet and an amniotic membrane sheet composite.

The sheet may comprise amniotic membrane that has been formed, e.g. cut, into a predetermined configuration, e.g. a predetermined shape and size. Shape and size may in this respect be in respect of the outline and surface area of the sheet.

Typically, the size may be range in a range of from 1.5 cm × 1.5 cm to 8 cm × 8 cm.

The amniotic membrane may, in use, be physically applied to a wound, to contact injured tissue of the wound. The amniotic membrane may thus be configured as a dressing, a patch, a tissue graft (such as an allograft), suitable for application to injured tissue of a wound.

The umbilical cord blood serum may be umbilical cord blood serum isolated from umbilical cord blood of a human or other mammal. This includes umbilical cord blood from human or other mammalian placenta.

The serum may be in a diluted and purified form, for use. More specifically, the umbilical cord blood serum may be diluted in a balanced salt solution according to the required concentrations of growth factors, such as EGF-1 or those described later on in this specification.

The umbilical cord blood serum may be provided, optionally formulated, as a liquid, a suspension, a solution, drops, or a dried, e.g. freeze-dried, powder.

ACCORDING TO A SECOND ASPECT OF THE INVENTION THERE IS PROVIDED a medicament comprising amniotic membrane and umbilical cord blood serum for use in a method of therapy, in particular for use in a method of treating a wound, e.g. a tissue wound, more specifically a chronic or non-healing wound, in a human or other mammalian subject.

The medicament may be a medicament according to the first aspect of the invention.

The treatment may be treatment according to the method of the sixth aspect of the invention.

The wound may be a wound that has not fully or adequately healed, e.g. in response to an earlier treatment.

The wound may, for example, include a soft tissue wound, a burn, an ulcer (such as a diabetic ulcer or a venous ulcer), a surgical wound and a chronic wound.

In some cases, the subject may have a dysfunction in the wound healing process.

The medicament, according to the first or second aspects of the invention, may include growth factors and cytokines, typically at high levels.

“High levels” may include a level in a range of from 1 picograms per millilitre (pg/ml), up to levels as high as 50 000 pg/ml.

Such growth factors and cytokines may be provided by, i.e. included in, the umbilical cord blood serum. The umbilical cord blood serum may therefore be selected based on its content of desired growth factors and cytokines.

In particular, the umbilical cord blood serum may be selected to include growth factors and cytokines that increase the amniotic membrane’s wound healing activity, including inflammation, cellular proliferation and tissue remodelling. In this regard it has surprisingly been found that the combination of umbilical cord blood serum with amniotic membrane increases the wound healing activity in comparison to the amniotic membrane on its own, including quicker healing of wounds and decreased scarring. Thus, the growth factors and cytokines may promote wound healing activity initiated by the amniotic membrane.

In one embodiment of the invention, desired growth factors and cytokines may include one or more, typically all, of EGF (epidermal growth factor), HGF (hepatocyte growth factor), MIF (macrophage migration inhibitory factor), and SCGF-β (stem cell growth factor beta).

In another embodiment of the invention, desired growth factors and cytokines may include one or more, typically all, of EGF (epidermal growth factor), TGF (transforming growth factor), HGF (hepatocyte growth factor), VEGF (vascular endothelial growth factor), PDGF (platelet derived growth factor), FGF (fibroblast growth factor), KGF (keratinocyte growth factor), MIF (macrophage migration inhibitory factor), SCGF (stem cell growth factor), CTACK (cutaneous T-cell attracting chemokine), MIG (monokine induced by gamma interferon), SDF (stromal cell-derived factor), NGF (nerve growth factor) and vitamin A.

In another embodiment of the invention, the growth factors and cytokines may include one or more, typically all, of IL-1α (interleukin-1 alpha), IL-3 (interleukin-3), M-CSF (macrophage colony stimulating factor), IL-12p40 (interleukin-12 subunit p40), IL-16 (interleukin-16), MIF (macrophage migration inhibitory factor), IFN-α2 (interferon-alpha 2), IL-18 (interleukin-18), MIG (monokine induced by gamma interferon), MCP-3 (monocyte chemotactic protein 3), CTACK (cutaneous T-cell attracting chemokine), SCF (Skp, Cullin, F-box containing complex), TNF-β (tumour necrosis factor-beta), GROα (growth-regulated oncogene-alpha), SCGF-β (stem cell growth factor-beta), b-NGF (nerve growth factor-beta), HGF (hepatocyte growth factor), SDF-1α (stromal cell-derived factor-1 alpha), IL-2Rα (interleukin-2 receptor alpha), LlF (leukemia inhibitory factor) and TRAIL (tumor necrosis factor (TNF)-related apoptosis inducing ligand.

In another embodiment of the invention, the growth factors and cytokines may include one or more, typically all, of IL-3, M-CSF, IL-16, MIF, IL-18, MIG, MCP-3, CTACK, SCF, GROα, SCGF-β, HGF, SDF-1α, IL-2Rα, LlF and TRAIL.

The medicament, according to the first or second aspects of the invention, may include at least one excipient, a pharmaceutical carrier, a diluent, a preservative, a stabilizer, and/or an additional therapeutic compound.

The medicament, according to the first or second aspects of the invention, may be stored frozen or may be stored at room temperature.

ACCORDING TO A THIRD ASPECT OF THE INVENTION THERE IS PROVIDED amniotic membrane for use in a method of therapy, in particular in a method of treating a wound, typically a chronic or non-healing wound, in a subject, wherein the amniotic membrane is for application to the wound in combination with umbilical cord blood serum.

ACCORDING TO A FOURTH ASPECT OF THE INVENTION THERE IS PROVIDED umbilical cord blood serum for use in a method of therapy, in particular in a method of treating a wound, typically a chronic or non-healing wound, in a subject, wherein the umbilical cord blood serum is for application to the wound in combination with an amniotic membrane.

The amniotic membrane, according to the third and fourth aspects of the invention, may be as hereinbefore characterised with reference to the first and second aspects of the invention.

Thus, the amniotic membrane may be amniotic membrane isolated from human or other mammalian placenta, may be dehydrated to preserve its extracellular matrix, and its epithelial cell layer may have been removed, or the amniotic membrane may be cryopreserved, in which case its epithelial cell layer may have been retained.

Furthermore, as hereinbefore characterised, the amniotic membrane may be provided as a dressing, a patch, a tissue graft, a sheet or similar.

Also, the umbilical cord blood serum, according to the third and fourth aspects of the invention, may be as hereinbefore characterised.

Thus, the umbilical cord serum may be umbilical cord serum isolated from umbilical cord blood from human or other mammalian placenta, and it may be in a diluted and purified form, for use.

The umbilical cord blood serum may be provided, optionally formulated, as a liquid, a suspension, a solution, drops, or a dried powder.

The wound may be a wound that has not healed fully or adequately in response to an earlier treatment, and may include a soft tissue wound, a burn, an ulcer (such as a diabetic ulcer or a venous ulcer), a surgical wound and a chronic wound.

In some cases, the subject may have a dysfunction in their wound healing process.

The amniotic membrane and the umbilical cord blood serum, as characterised with reference to any of the first to fourth aspects of the invention may be for administration, more specifically application, simultaneously, in sequence, at different time intervals, or may be for repeated administration, e.g. over several hours, days, or weeks.

Also as characterised with reference to any one or more of the above aspects of the invention, the amniotic membrane may be provided, optionally prepared, in the manner characterised, and thus for application to a surface of a wound.

Such application may, in use, for example be by gluing or suturing.

The umbilical cord serum may be formulated for administration, more specifically application, to the applied amniotic membrane, e.g. in liquid or solid, such as powder (e.g. a freeze-dried powder), form.

ACCORDING TO A FIFTH ASPECT OF THE INVENTION THERE IS A PROVIDED a kit comprising dehydrated or cryopreserved amniotic membrane and umbilical cord blood serum, as respectively hereinbefore characterised with reference to the first to fourth aspects of the invention, for use, in combination, in a method of therapy, in particular in a method of treating a wound, typically a chronic or non-healing wound, in a subject.

The treatment may be treatment according to the method of the sixth aspect of the invention.

ACCORDING TO A SIXTH ASPECT OF THE INVENTION THERE IS PROVIDED a method of treating a wound, typically a chronic or non-healing wound, in a subject, the method comprising administering, more specifically applying, amniotic membrane and umbilical cord blood serum, as hereinbefore characterised with reference to the first to fourth aspects of the invention, to a wound in a subject, typically wherein the wound has not healed in response to an earlier treatment and/or wherein there is a dysfunction in the subject’s wound healing process.

Administering, more specifically applying, the amniotic membrane to the wound, may include locating the amniotic membrane such that it contacts the wound, specifically injured tissue of the wound, e.g. such that it covers the wound.

Administering, more specifically applying, the umbilical cord blood serum to the wound may include contacting the wound, specifically injured tissue of the wound, with the umbilical cord blood serum, e.g. by contacting the amniotic membrane, as applied to the wound, with umbilical cord blood serum.

Application of the amniotic membrane and the umbilical cord blood serum to the wound may be simultaneous or separate, e.g. sequential.

ACCORDING TO A SEVENTH ASPECT OF THE INVENTION there is provided a method of preparing dehydrated amniotic membrane, the method comprising
isolating amniotic membrane from human or other mammalian placental tissue, including from a chorion;
sterilising the isolated amniotic membrane in an antibiotic and antimycotic solution;
dehydrating the sterilised amniotic membrane; and
sterilising the dehydrated amniotic membrane using gamma irradiation.

The dehydrated amniotic membrane this produced may be an amniotic membrane as characterised with reference to the first to sixth aspects of the invention.

The dehydrated amniotic membrane thus produced, may be one embodiment of an amniotic membrane as provided for in the first to sixth aspects of the invention.

It has been found that, through the method, the extracellular matrix of the amniotic membrane is preserved in its dehydrated form.

Also, it was found that the amniotic membrane thus produced may be stored at room temperature without negatively affecting its usefulness in the invention, as hereinbefore characterised with reference to the first to sixth aspects of the invention.

ACCORDING TO AN EIGHTH ASPECT OF THE INVENTION THERE IS PROVIDED a method of preparing cryopreserved amniotic membrane for use in any of the first to sixth aspects of the invention, the method comprising isolating the amniotic membrane from human or other mammalian placental tissue; sterilising the amniotic membrane in an antibiotic and antimycotic solution; and freezing the amniotic membrane at -80° C. on a nitrocellulose membrane carrier.

The cryopreserved amniotic membrane thus produced, may be one embodiment of an amniotic membrane as provided for in the first to sixth aspects of the invention.

ACCORDING TO A NINTH ASPECT OF THE INVENTION IS PROVIDED use of amniotic membrane and umbilical cord blood serum in the manufacture of a medicament for treating a wound in a human or other mammalian subject.

The amniotic membrane and umbilical cord blood serum may be as hereinbefore characterised with reference to the first to eighth aspects of the invention.

Furthermore, treating a wound using the medicament so produced, may be as hereinbefore characterised with reference to the first to eighth aspects of the invention.

### EXAMPLES

The invention will now be described in more detail with reference to the examples hereunder, and the accompanying drawings.

In the drawings

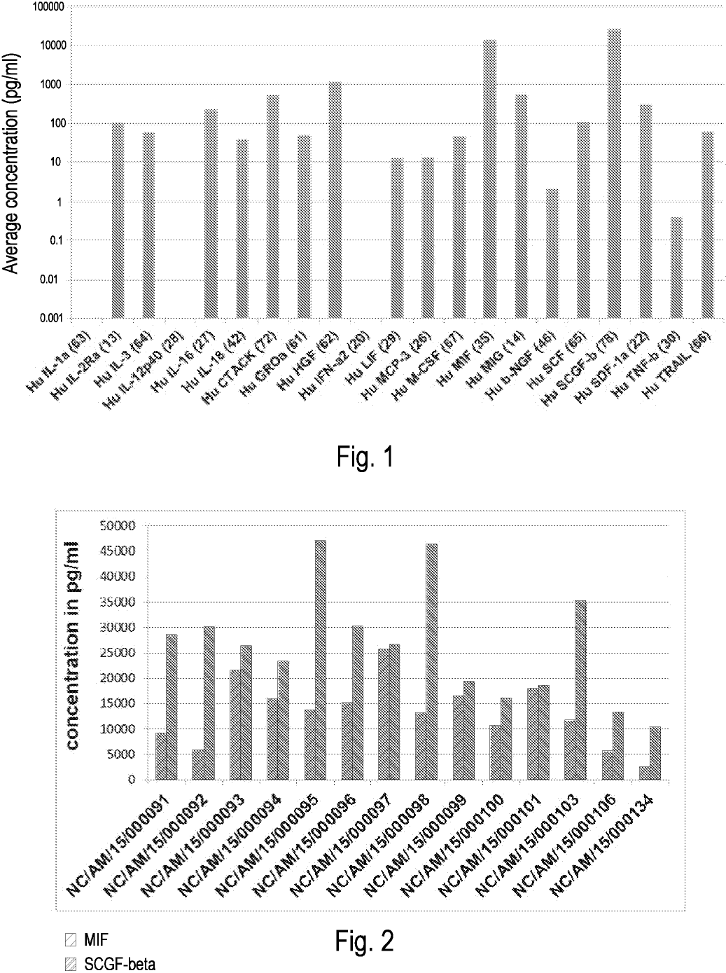

FIG. 1 shows, for Example 1, concentrations of cytokines and growth factors in cord blood serum;

FIG. 2 shows, for Example 1, concentrations of MIF and SCGF-beta in cord blood serum;

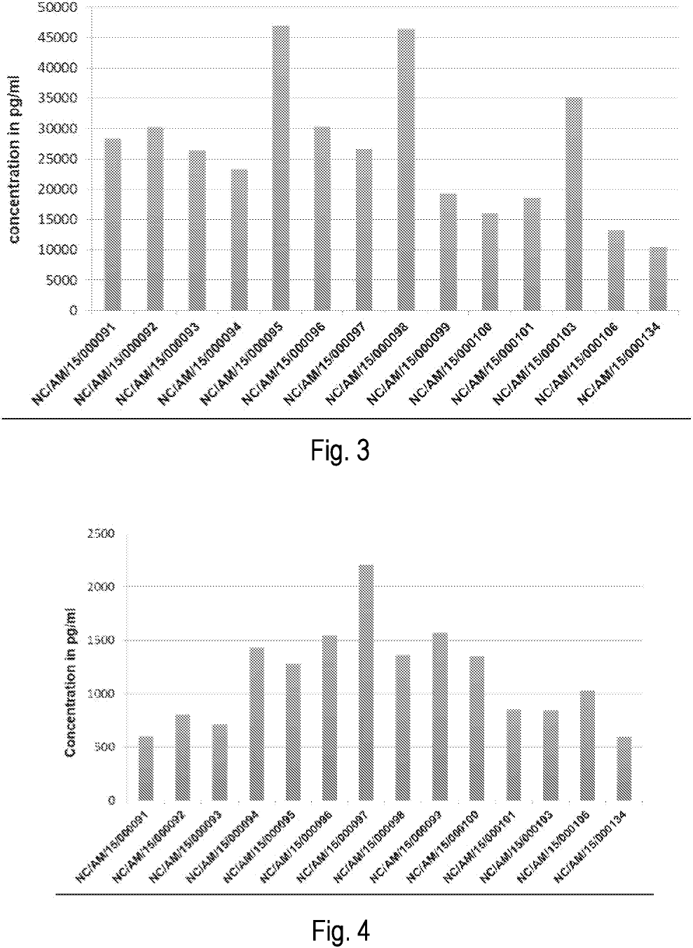

FIG. 3 shows, for Example 1, concentrations of SCGF-beta in cord blood serum;

FIG. 4 shows, for Example 1, concentrations of HGF in cord blood serum;

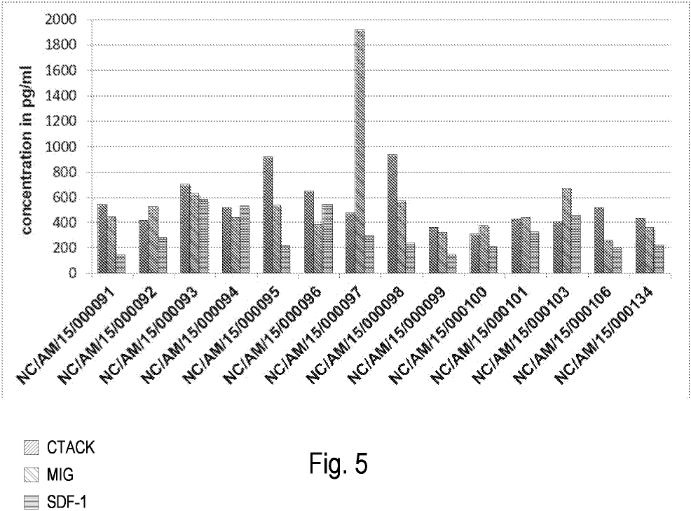

FIG. 5 shows, for Example 1, concentrations of CTACK, MIG and SDF-1 in cord blood serum;

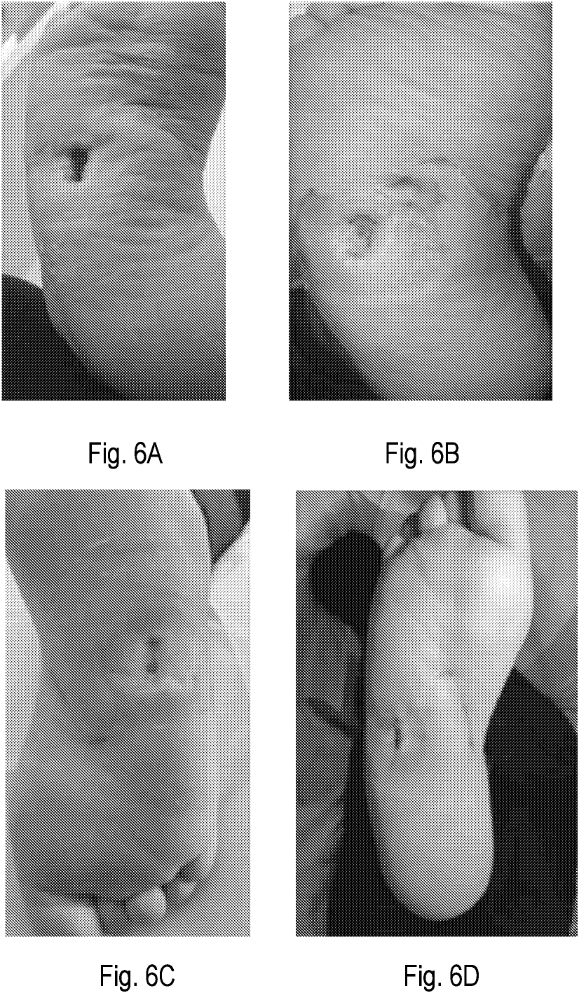

FIG. 6 shows, for Example 2, treatment of a spider bite wound with (A) the wound before treatment, (B) four days post treatment, (C) five weeks post treatment, (D) fifteen weeks post treatment; and

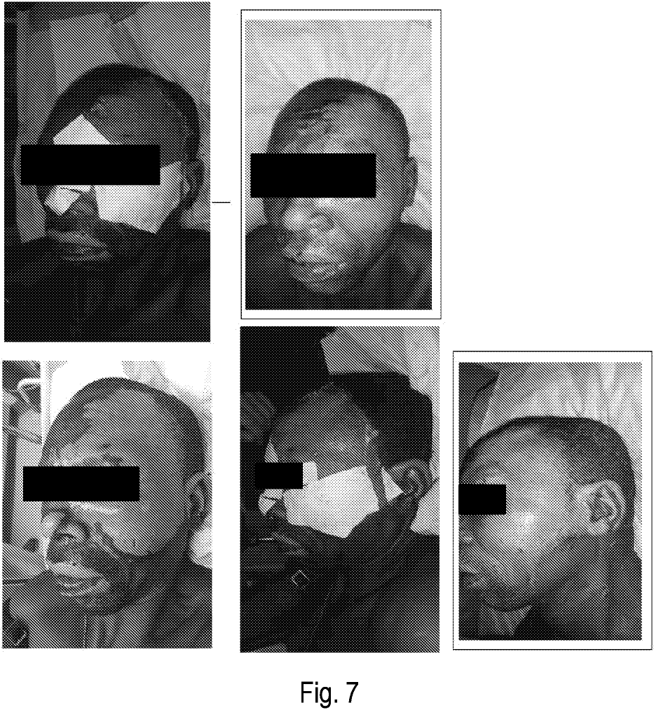

FIG. 7 shows, for Example 3, treatment of a partial burn wound using dehydrated prepared amniotic membrane.

### EXAMPLE 1

### Materials and Methods

### Umbilical Cord Blood Serum Composition

### Placental Donation and Umbilical Cord Blood Collection

Placentas were collected from human female subjects. These mothers had uncomplicated pregnancies and gave their informed consent for the retrieval of umbilical cord blood and amniotic membrane from the placenta for therapeutic and research purposes. Samples of the mother’s blood were taken and tested for HIV, Hepatitis B and C, HTLV I and II, CMV and syphilis. Umbilical cord blood was collected by direct cannulation of cord blood into a component blood transfer bag/ 50 ml centrifuge/falcon tube, with no anti-coagulants or preservatives, taking all sterile precautions after the infant was removed and the umbilical cord clamped. The blood was drained from the placenta by gravitation.

### Laboratory Processing

The blood was stored at 4° C. for 2 to 24 hours. The clotted blood was centrifuged for 60 minutes at 3500 rpm, at room temperature. The serum fraction (supernatant) was removed from the clotted pellet and transferred to a sterile 50 ml falcon tube, using aseptic techniques. The serum was diluted with Balanced Salt Solution (BSS) to make a 20% dilution. The diluted cord blood serum was centrifuged for 10 minutes at 3500 rpm, at room temperature following which it was filter sterilized through a 0.20 µm filter.

After filter sterilization 4.5 ml aliquots of the diluted serum were transferred to 5.0 ml dropper bottles. The pH and ABO blood grouping of the serum was tested using standard procedures.

Umbilical cord blood serum may be freeze-dried and processed to a powder form which is stable at room temperature.

Umbilical cord blood serum is stored at -80° C. for long term storage. For use, it is thawed and stored at 4-8° C.

### Analysis of Contents of Umbilical Cord Blood Serum

In order to gain insight into the mechanism whereby umbilical cord blood serum (as found in the Next Biosciences OptiSerum™ product) mediates its regenerative effect, the umbilical cord serum from 14 donors was analyzed for the presence of 21 cytokines and growth factors.

A Bio-Plex Pro Human Cytokine Standard 21-plex (BioRad; Group II) and a BioPlex 200 multiplex system was utilized to assess cytokine levels in umbilical cord blood from 14 donors. Dilutions of standards were as directed by the manufacturer, while all dilutions of cord blood serum (using the manufacturer’s sample diluent) were at 20% (as per OptiSerum™). The concentration of the following cytokines and growth factors in umbilical cord serum (IL-1α, IL-3, M-CSF, IL-12 p40, IL-16, MIF, IFN-α2, IL-18, MIG, MCP-3, CTACK, SCF, TNF-β, HGF, SDF-1α, IL-2Rα, LlF, TRAIL) were determined as per the manufacturer’s instructions. Eighteen of these were found to be present in the samples tested. The average levels of 16 cytokines and growth factors range from 10 - 50 000 pg/ml (many of these cytokines are either immune modulators or stem cell factors), while two are detected between 0.4 and 2 pg/ml. Three pro-inflammatory cytokines (IL-1α, IL-12 p40; IFN-α2) are not detectable in umbilical cord serum.

### Amniotic Membrane

### Mechanical Removal of the Amniotic Membrane from the Chorion and Other Placental Tissue

Using aseptic techniques, placenta donated by human female subjects who had uncomplicated pregnancies were transferred from sterile transport bags to the stainless steel dish with 500 ml cold saline. The placenta was rinsed to remove any blood. The amniotic membrane was mechanically removed from the chorion by pulling the amniotic membrane off by hand towards the umbilical cord and the membrane was cut loose from the cord. The membrane was rinsed in 200 ml saline.

The chorionic membrane was wiped from the amniotic membrane with sterile gauze and saline. The membrane was rinsed repeatedly with saline, to remove all residual tissue and to keep the membrane wet. The membrane was transferred to 1% antibiotic/antimycotic : DMEM solution and incubated at 2 - 8° C. for 48 - 72 hrs.

### Dehydrated Amniotic Membrane

The amniotic membrane was wiped to remove all excess chorion and antibiotic/antimycotic solution and rinsed in sterile saline. The membrane was incubated in Trypsin/EDTA solution for 2 hours 30 minutes. The amniotic membrane was removed from the Trypsin/EDTA solution and washed with DMEM. Gauze wipes were used to remove the amniotic epithelial cell layer following which the membrane was rinsed repeatedly with saline to remove all residual tissue and to keep the membrane wet. The amniotic membrane was stretched out on a clean dry surface in a Bio-Flow cabinet and left until completely dry and then packaged and sterilized using Gamma irradiation. Dehydrated amniotic membrane is stored at room temperature.

### Cryopreserved Amniotic Membrane

The amniotic membrane was wiped to remove all excess chorion and antibiotic/antimycotic solution and rinsed in sterile saline. The membrane was incubated in Trypsin/EDTA solution for 2 hours 30 minutes. The membrane was removed from the Trypsin/EDTA solution and washed with DMEM. The amniotic membrane was stretched out on a nitrocellulose membrane carrier with the stromal side of the membrane facing onto the nitrocellulose paper. DMEM: 87% Glycerol (1:1 ratio (v/v)) was added to the amniotic membrane before freezing at -80° C. Cryopreserved amniotic membrane is stored at -20° C. For use, it is thawed at room temperature and washed in saline to remove the glycerol cryoprotectant.

### Treatment of Soft Tissue Wounds

An appropriately sized dehydrated or cryopreserved amniotic membrane dressing was applied so that it covers the enter wound. Following this, 4 drops of umbilical cord blood serum was applied to the wound, directly onto the amniotic membrane wound dressing. A further 4 drops of umbilical cord blood serum were placed on the wound every 4 days until the wound was fully healed. If required a new amniotic membrane dressing was placed on the wound every 7 days. The dehydrated membrane was found to attach easily to the wound surface and to absorb fluids from the wound.

### Results

### Analysis of Umbilical Cord Blood Serum

The product umbilical blood cord serum (OptiSerum™) was analyzed to determine the concentration of various growth factors and cytokines. Values shown in the results take the dilution of umbilical cord blood serum into account and represent the levels in the original umbilical cord blood serum. The cytokines successfully tested are shown in Table 1 below. ICAM-1 and VCAM-1 standards were not successfully detected.

Table 1

List of growth factors and cytokines included in the BioPlex Group II Human Cytokine Assay

| | | |
|---|---|---|
| IL-1α interleukin-1 alpha | IL-3 interleukin-3 | M-CSF macrophage colony stimulating factor |
| IL-12 p40 interleukin-12 subunit p40 | IL-16 interleukin-16 | MIF macrophage migration inhibitory factor |
| IFN-α2 interferon-alpha 2 | IL-18 interleukin-18 | MIG monokine induced by gamma interferon |
| MCP-3 monocyte chemotactic protein 3 | CTACK cutaneous T-cell attracting chemokine | SCF Skp, Cullin, F-box containing complex |
| TNF-P tumour necrosis factor-beta | GROα growth-regulated oncogene-alpha | SCGF-β stem cell growth factor-beta |
| b-NGF nerve growth factor-beta | HGF hepatocyte growth factor | SDF-1α stromal cell-derived factor-1 alpha |
| IL-2Rα interleukin-2 receptor alpha | LIF leukemia inhibitory factor | TRAIL tumor necrosis factor (TNF)-related apoptosis inducing ligand |

Sixteen growth factors and cytokines (out of the 21 tested) are present at significant levels in the cord blood samples tested (FIG. 1). Average levels range from 10 - 50 000 pg/ml. Many of these cytokines are either immune modulators or stem cell factors. Detectable levels of the remaining five cytokines (IL-1α; IL-12 p40; IFN-α2; TNF-β; β-NGF) are negligible.

Within the group of 21 cytokines, 3 are present at very high levels and specifically at concentrations greater than 1000 pg/ml (in other words, in the ng/ml range). These are Macrophage migration inhibitory factor (MIF), Stem cell growth factor beta (SCGF-β) and Hepatocyte growth factor (HGF). The concentration of these factors in each of the cord blood samples is shown in FIGS. 2-4. The concentration of each protein varies considerably between the 14 samples tested i.e. 2000-25 000 pg/ml (MIF); 10 000 -50 000 pg/ml (SCGF-beta); 500 - 2000 pg/ml (HGF).

Three additional cytokines are expressed at high levels (FIG. 5), namely Cutaneous T-cell attracting chemokine (CTACK), Monokine induced by gamma IFN (MIG) and Stromal cell-derived factor 1 (SDF-1). The concentration of these factors generally ranges between 100 pg/ml and 1000 pg/ml, with one sample (NC/AM/15/000097) containing 2000 pg/ml of MIG (FIG. 5). It is understood that epidermal growth factor (EGF), hepatocyte growth factor (HGF), Macrophage migration inhibitory factor (MIF) and Stem cell growth factor beta (SCGF-β) would be present at high levels in umbilical cord blood serum as these growth factors are associated with stem cell growth and development.

### Example 2

### Treatment of Spider Bite Wound With Prepared Amniotic Membrane (Amniomatrix™) and Umbilical Cord Blood Serum (Optiserum™)

The subject is a 24 year old female patient with Spina Bifida suffering from a non-healing wound on her bottom right foot and difficulty walking due to abnormal weight distribution on her feet. In March 2013, the patient developed cellulitis after a suspected spider bite on the base of her right foot. The infection was treated successfully, however, the wound did not heal and instead became necrotic. She had multiple attempts at healing the wound with wound healing care products, surgery and had undergone skin grafts and tendon transplants, none of which were successful.

A 4×4 cm dehydrated amniotic membrane dressing accompanied by 4 drops of umbilical cord serum was applied to the wound every four days for a 15 week period. From the initial dressing, quite a significant improvement was noted with the wound getting narrower, superficial and clean. Over the weeks, the wound healed remarkably with prominent wound healing stages observed i.e. less callous formation, granulation, keratin formation, epithelialization and a tougher outer surface, thus leading to complete wound closure and healing (FIGS. 6A-D). At 4 days post treatment (FIG. 6B) the wound was narrower, superficial and clean. At 5 weeks post treatment (FIG. 6C), there was good granulation observed and wound contraction. At 15 weeks post treatment (FIG. 6D), there was good epithelialization, a tougher outer surface and wound closure.

### Example 3

### Treatment of Partial Burn Wound to the Face With Dehydrated Prepared Amniotic Membrane (Amniomatrix™)

A 32 year old male subject patient presented with partial thickness burns to the face. The injury occurred on 22 Feb. 2016. Dehydrated prepared amniotic membrane was applied to the subject’s forehead. As a comparison, Nanotrix^{®} (Southern Medical (Pty) Ltd, Southern Implants Office park, 1 Albert Road, Irene, 0062) was applied to the other areas. Nanotrix is a temporary synthetic skin substitute used for treating burns. Simple chloromycetin ointment was applied as an antibiotic over the membranes and there was no sepsis of the wounds. The wound covered by amniotic membrane healed by 25 Feb. 2016 whereas the wound covered by Nanotrix healed by 27 Feb. 2016 (FIG. 7) demonstrating that the amniotic membrane showed improved efficacy over the synthetic Nanotrix product in healing of the burn wound in that the same level of healing was achieved two days earlier with the amniotic membrane than the Nanotrix product. This outcome supported favouring a biologic material over a synthetic material in the healing of wounds, which the present invention inventively provides for with the additional employment of umbilical cord blood serum.

### Discussion

AMNIOTIC MEMBRANE ALLOGRAFTS have been used as a biologic therapy to promote soft tissue healing in ophthalmic and other peripheral wounds. A primary characteristic of amniotic membrane that assists with wound healing is the provision of a scaffold of pre-formed extracellular matrix composed of natural collagen and other proteins. Dehydrated amniotic membrane reduces wound pain and reduces the level of exudate produced by the wound.

However, it was found by the inventors that many growth factors and cytokines that facilitate the wound healing process through migration of biologically active cells to the wound and stimulation of the proliferation, differentiation and growth of these cells, are not present, due to the processing and storing of the amniotic tissue.

It was surprisingly found, by the inventors, that this lack of growth factors and cytokines can be effectively be corrected by co-administration of umbilical cord blood serum, in accordance with the invention.

It is demonstrated, in accordance with the invention, that umbilical cord blood serum contains many growth factors and cytokines that are responsible for cell migration, proliferation and differentiation. Thus, co-administration of amniotic membrane grafts and umbilical cord blood serum was surprisingly found to improve the dysfunction of the inflammation, cellular proliferation, and tissue remodelling stages of the wound healing process and promotes quicker healing of wounds with decreased scarring.

It was therefore surprisingly found that a combination of umbilical cord blood serum and amniotic membrane, according to the invention, enhances wound healing activity in comparison to the amniotic membrane on its own, including quicker healing of wounds and decreased scarring.

The co-administration of amniotic membrane and umbilical cord blood serum according to the invention, was therefore found to be particularly used for the treatment of “treat and heal” soft tissue wounds such as spider bites, burns and diabetic ulcers even when these wounds do not respond satisfactorily to other earlier-administered therapies including wound healing care products, surgery, skin grafts and tendon transplants.

Furthermore, dehydrated amniotic membrane was found to stick to wound surfaces very effectively and also to absorb fluids from the wound. It was further found to reduce pain arising from the wound and to reduce the level of exudate produced by the wound.

1-9. (canceled)

10. A method of treating a tissue wound in a human or other mammalian subject comprising the steps of:
applying an amniotic membrane to an injured tissue of the wound to contact the injured tissue of the wound; and
contacting an amniotic membrane with an umbilical cord blood serum;
wherein the amniotic membrane is isolated from human or other mammalian placenta;
wherein the amniotic membrane is dehydrated or cryopreserved and is in a form selected from
a sheet of amniotic membrane, or
an amniotic membrane sheet composite, comprising multiple overlain sheets of amniotic membrane; and
wherein the umbilical cord blood serum is isolated from umbilical cord blood of a human or other mammal separately of each other.

11. The method according to claim 10, wherein the sheet of amniotic membrane comprises an amniotic membrane that has been formed into a predetermined shape and size.

12. The method according to claim 10, wherein the umbilical cord blood serum is selected to include growth factors and cytokines that include one or more or all of epidermal growth factor, hepatocyte growth factor, macrophage migration inhibitory factor (MIF), and stem cell growth factor beta (SCGF-β) that promote wound healing activity initiated by the amniotic membrane.

13. The method according to claim 10, wherein the umbilical cord blood serum is selected to include growth factors and cytokines that include one or more or all of IL-1a, IL-3, M-CSF, IL-12 p40, IL-16, MIF, IFN-a2, IL-18, MIG, MCP-3, CTACK, SCF, TNF-β, GROa, SCGF-β, b-NGF, HGF, SDF-1a, IL-2Ra, LIF, and TRAIL.

14. The method according to claim 13, wherein the umbilical cord blood serum is selected to include growth factors and cytokines that include one or more or all of IL-3, M-CSF, IL-16, MIF, IL-18, MIG, MCP-3, CTACK, SCF, GROa, SCGF-β, HGF, SDF-1a, IL-2Ra, LIF, and TRAIL.

15. The method according to claim 10, wherein the umbilical cord blood serum includes growth factors and cytokines at a level in a range of from 1 pg/ml to 50000 pg/ml.

16. The method according to claim 10, wherein the umbilical cord blood serum is provided as a liquid, a suspension, a solution, drops, or a dried powder.

17. The method according to claim 10, wherein the tissue wound is a soft tissue wound, a burn, an ulcer, a surgical wound, or a chronic wound that has not healed fully in response to an earlier treatment.

18. A method of treating a wound in a human or other mammalian subject, the method comprising applying a sheet of dehydrated or cryopreserved amniotic membrane to the wound and contacting the amniotic membrane with umbilical cord blood serum, wherein the amniotic membrane was isolated from human or other mammalian placenta.
